(19) United States
(12) Patent Application Publication
(10) Pub. No.: US 2024/0093148 A1
(43) Pub. Date: Mar. 21, 2024

(54) METHOD FOR DIFFERENTIATING HUMAN INDUCED PLURIPOTENT STEM CELLS INTO OLIGODENDROCYTES, AND KIT AND USE

(71) Applicant: ALLIFE MEDICINE (ZHUHAI) LIMITED, Zhuhai (CN)

(72) Inventors: Lida WU, Zhuhai (CN); Yuchun GU, Zhuhai (CN)

(73) Assignee: ALLIFE MEDICINE (ZHUHAI) LIMITED, Zhuhai (CN)

(21) Appl. No.: 18/521,956

(22) Filed: Nov. 28, 2023

Related U.S. Application Data
(63) Continuation of application No. PCT/CN2022/088517, filed on Apr. 22, 2022.

(30) Foreign Application Priority Data
Aug. 5, 2021 (CN) 202110894940.X

(51) Int. Cl. C12N 5/079

(52) U.S. Cl. CPC C12N 5/0622; C12N 2500/32; C12N 2500/38; C12N 2500/44; C12N 2501/01; C12N 2501/105; C12N 2501/12; C12N 2501/13; C12N 2501/135; C12N 2501/33; C12N 2501/395; C12N 2501/41; C12N 2501/999; C12N 2506/45

(57) ABSTRACT

Provided are a method for differentiating human induced pluripotent stem cells into oligodendrocytes, and a kit and the use. The method comprises culturing stem cells by means of using at least one of the following culture media: a neural induction complete culture medium, an N2 culture medium, a B27 culture medium and an OPC maturation culture medium. More preferably, the induction of oligodendrocytes by an OPC maturation culture medium with puerarin increases the number of oligodendrocytes by 30% compared with a culture medium without puerarin.

### FIELD

The present invention relates to the field of biotechnology, in particular to a method, kit, and application for differentiating human induced pluripotent stem cells into oligodendrocytes.

### BACKGROUND

Oligodendrocyte precursor cells (OPC) are central nervous system cells present in vertebrates, forming myelin sheaths that surround nerve fibers, playing a crucial role in the transmission of neural signals and the nutrition and protection of nerve fibers. Oligodendrocytes produce lipid rich layered myelin sheaths, which differentiate and mature to form myelin sheaths, covering neuronal axons and generating limited electrical insulation segments to maximize action potential conduction velocity. Myelin is also important for axonal integrity and survival, and it has been shown that even small changes affecting oligodendrocyte metabolism can lead to neurodegeneration. The process of myelination is particularly important in humans, as the human brain has a high concentration of myelinated neurons (white matter), and myelination persists after birth and lasts throughout a lifetime. This indicates that oligodendrocytes not only provide inert insulation, but also myelination is a dynamic process that affects cognitive function and even behavior. Multiple sclerosis (MS), adrenoleukodystrophy, white matter ablation disease, Pelizaeus Merzbach disease, and cerebral white matter malnutrition are examples of demyelinating or myelination disorders. In addition, the key role of oligodendrocytes is being demonstrated in many other neurological disorders and neurodegenerative conditions, including amyotrophic lateral sclerosis, Huntington's disease, Alzheimer's disease, and schizophrenia.

The advancement of neural stem cell biology and clinical applications has provided possibilities for the treatment of intractable neurological diseases. At the same time, improving the survival rate of implanted OPCs and improving their microenvironment is also crucial for using neural stem cells to treat neurological diseases. OPC derived from humans not only helps to better understand the function of oligodendrocytes and axon neuron interactions, but also provides an essential tool for myelin repair research and drug therapy.

Human inducible pluripotent stem cells (iPSCs) can be induced in vitro to form neural stem cells (NSCs), which in turn produce different lineages of neurons and glial cells, such as oligodendrocyte precursor cells (OPCs) or astrocytes. IPSCs can also be directly induced into OPCs, but current technology requires a long time and the efficiency is low.

### SUMMARY

The present invention provides an improved method for inducing pluripotent stem cells to differentiate into oligodendrocytes mediated by small molecules and other factors. The present 10 invention is based on the same improved method for inducing OPCs by iPSC through NSC, and the experiment confirms that puerarin increases the induction efficiency and quantity of induced pluripotent stem cells to differentiate into oligodendrocytes, and can increase the number of oligodendrocytes by 30% in the later stage of induction.

### Method

In the first aspect, the present invention provides a method for inducing oligodendrocytes, which includes culturing stem cells using at least one of the following culture media:
1) Neural induction complete culture medium;
2) N2 culture medium;
3) B27 culture medium;
4) OPC mature culture medium.

Preferably, the method involves sequentially using neural induction complete culture medium, N2 culture medium, B27 culture medium, and OPC mature culture medium for stem cell culture.

Preferably, the time for using the neural induction complete culture medium is 4-10 days; preferably, 7 days; more preferably, days 0-7.

Preferably, the time for using the N2 culture medium is 1-6 days; preferably, 3 days; more preferably, days 8-11.

Preferably, the time for using the B27 culture medium is 4-10 days; preferably, 7 days; more preferably, days 12-19.

Preferably, the time for using the OPC mature culture medium is at least 7 days; preferably, at least 10 days; more preferably, the 20th to 30th days.

Preferably, the culture is a cell culture of any cell derived from humans, orangutans, monkeys, horses, cows, sheep, pigs, donkeys, camels, dogs, rabbits, cats, rats, mice, fish, birds, or insects.

Preferably, the stem cells include one or more of ESC (embryonic stem cells), inducible pluripotent stem cells (iPSC), embryoid bodies, cellular hematopoietic stem cells, neural stem cells, mesenchymal stem cells, skin stem cells, adipose stem cells, and umbilical cord blood stem cells.

More preferably, the stem cells are iPSCs.

Preferably, the iPSC cells can be commercialized cell lines or induced by donor cells, including villous cells, skin (fibroblasts and keratinocytes), amniotic fluid, extracellular tissue (placenta and umbilical cord), umbilical cord blood, periosteum, dental tissue, adipose tissue, neural stem cells, liver cells, mesenchymal stem cells, peripheral blood cells, mammary epithelial cells, and adipose stem cells, One or more of the umbilical cord matrix and placenta.

In one embodiment, the neural induction complete culture medium contains a first small molecule compound combination.

In one embodiment, the neural induction complete culture medium consists of a first basic culture medium, non-essential amino acids, glutamine, reducing agent, and a first small molecule compound combination.

Preferably, the first basic culture medium, second basic culture medium, third basic culture medium, and fourth basic culture medium described in the present invention independently include TeSR-E8, mTESR1, E8, and Essential 8 ™ Medium, Dulbecco's Modified Eagle's Medium, Minimum Essential Medium, BME, F-10, F-12 α-Minimum essential medium (α-MEM, α-Minimal Essential Medium, G-MEM, Glasgow's Minimal Essential Medium, IMPM (IMDM, Discover's Modified Dulbecco's Medium), AnnioMax, New Second Generation Amniotic Fluid Medium (Amino Max II complete Medium, Gibco, NewYork, USA), Chang's medium, Mesem Cult-XF medium (STEMCELL Technologies, Vancouver, Canada), RPMI1640, Ham's F12, DMEM/F12 Ham's F-12K Medium, Hepato ZYME-SFM, William's E Medium, Waymouth's Medium, or Hepatocyte Culture Medium.

Preferably, the first basic culture medium, the second basic culture medium, the third basic culture medium, and the fourth basic culture medium of the present invention are DMEM/F12.

Preferably, the reducing agent described in the present invention includes but is not limited to β-mercaptoethanol (2-mercaptoethanol), dithiothreitol, dithioerythritol, reduced glutathione, cysteine, thiocarbamate, sodium dithiosulfite, ascorbate, tin dichloride, or sodium borohydride.

Preferably, the glutamine described in the present invention uses GlutaMAX-I, which is a cell culture additive that can directly replace L-glutamine in cell culture medium.

Preferably, the non-essential amino acids described in the present invention include alanine, arginine, aspartic acid, cystine, proline, and tyrosine.

Preferably, the neural induction complete culture medium consists of 98% DMEM/F-12 culture medium, 1% non-essential amino acid, 1% GlutaMAX-I, 2-mercaptotothanol, and a first small molecule compound combination.

Preferably, the neural induction complete culture medium consists of 98% DMEM/F-12 culture medium (Life Technologies, cat. no. 11039021), 1% non-essential amino acid (Life Technologies, cat. no. 11140-050), 1% GlutaMAX-I (Life Technologies, cat. no. 35050061), 2-Mercaptotothanol (Gibco, 31350010), and a first small molecule compound combination. The parentheses represent the manufacturer and item number of the product.

Preferably, the first small molecule compound combination includes at least one of insulin, TGF-β signaling pathway inhibitors, BMP signaling pathway inhibitors, and agonists of RAR nuclear receptors.

Preferably, the TGF-β Signal pathway inhibitors include SB431542, Dorsomorphin, A83-01, LDN193189, RepSox, SB 525334, SB-505124, BMP signaling inhibitor sb4, GW788388, ITD-1, LSKL, SD-208, LDN-212854, K02288, LDN-214117, R-268712, SM16, A 77-01, ALK2-IN-2, PD-161570, pm26, TGF-β1 peptide TFA, Isosaponarin, BIO-013077-01.

Preferably, the TGF-β signaling pathway inhibitor selects SB431542; preferably, its article number is medchemexpress, HY-10431.

Preferably, the working concentration of SB431542 is 10 μM.

Preferably, the BMP signaling pathway inhibitors include Dorsomorphin, A83-01, LDN193189, RepSox, SB525334, DMH-1, SB-505124, BMP signaling inhibitor sb4, GW788388,

ITD-1, LSKL, SD-208, LDN-212854, K02288, LDN-214117, R-268712, SM16, A77-01, ALK2-IN-2, PD-161570, pm26, TGF-β peptide TFA, Isosaponarin, BIO-013077-01.

Preferably, the BMP signaling pathway inhibitor is LDN193189; preferably, its article number is medchemexpress, HY-12071.

Preferably, the working concentration of LDN193189 is 0.25 μM.

Preferably, the agonists of the RAR nuclear receptor include vitamin A acid, Rapamycin, 3-Methyladenine, Acetylcysteine, 5-Fluorouracil, Hydrocortisone, Docetaxel, Rosiglitazone, Estradiol, Melatonin, GW9662, Nicotinamide, Cytarabine, Isoprenaline hydrochloride, Prostaglandin E2, Acetaminophen, (−)-Epigallocatechin Gallate β-Nicotinamide mononucleotide, Calcitriol, DHEA, Liothyronine, NAD+, Luteolin, Thymidine, Docosahexaenoic Acid, Kaempferol, Palmitic acid, Cyclopamine, Genistein, L-Glutathione reduced.

Preferably, the agonist of the RAR nuclear receptor is vitamin A acid; preferably, its article number is medchemexpress, HY-14649.

Preferably, the working concentration of the vitamin A acid is 100 μM.

In one embodiment, the N2 culture medium comprises a second small molecule compound combination.

In one embodiment, the N2 culture medium consists of a second basic culture medium, non-essential amino acids, glutamine, reducing agent, N2 supplement, and a second small molecule compound combination.

The “N2 supplement” described in the present invention contains human transferrin Holo, recombinant insulin FullChain, Progesterone, Putrescine, Selenite, which can be commercialized or self-formulated.

Preferably, the N2 medium consists of 97% DMEM/F-12 medium, 1% non-essential amino acid, 1% GlutaMAX-I, 2-Mercaptotothanol, 1% N2 supplement, and a second small molecule compound combination.

Preferably, the N2 culture medium consists of 97% DMEM/F-12 culture medium (Life Technologies, cat. no. 11039021), 1% non-essential amino acid (Life Technologies, cat. no. 11140-050), 1% GlutaMAX-I (Life Technologies, cat. no. 35050061), 2-Mercaptoethanol (Gibco, 31350010), 1% N2 supplement (ThermoFisher, cat. no. 17502001), and a second small molecule compound combination. The parentheses represent the manufacturer and item number of the product.

Preferably, the second small molecule compound combination includes an agonist of RAR nuclear receptors and/or an activator of the Hedgehog signaling pathway.

Preferably, the agonist of the RAR nuclear receptor is consistent with the aforementioned.

Preferably, the Hedgehog signaling pathway activators include SAG, Cyclopamine, Purmorphamine, 20 (S)-Hydroxycholesterol, Halcinonide, Jervine, ALLO-2, IHR-Cy3.

Preferably, the Hedgehog signaling pathway activator is SAG; preferably, its article number is medchem express, HY-12848.

In one embodiment, the B27 culture medium comprises a third small molecule compound combination.

In one embodiment, the B27 culture medium consists of a third basic culture medium, non-essential amino acids, glutamine, reducing agent, N2 supplement, B27 supplement, and a third small molecule compound combination.

The “B27 supplement” mentioned in the present invention contains biotin and DL-α-DL Alpha tocopherol acetate, DL-α-DL Alpha Tocopherol, BSA (fatty acid free Fraction V), Catalase, Human Recombinant Insulin, Human Transferrin, Superoxide Dismutase, Corticosterone, D-Galactose, Ethanolamine HCl Reduced Glutathione L-Carnitine HCl Linoleic Acid, Linolenic Acid, Progesterone, Putrescine 2HCl Sodium Selenite, and T3 triodo I-thyronine, which can be commercially available or prepared by oneself

Preferably, the B27 culture medium consists of 95% DMEM/F-12 culture medium, 1% non-essential amino acid, 1% GlutaMAX-I, 2-Mercaptotothanol, 1% N2 supplement, 2% B27 supplement, and a third small molecule compound combination.

Preferably, the B27 culture medium consists of 95% DMEM/F-12 culture medium (Life Technologies, cat. no. 11039021), 1% non-essential amino acid (Life Technologies, cat. no. 11140-050), 1% GlutaMAX-I (Life Technologies, cat. no. 35050061), 2-Mercaptoethanol (Gibco, 31350010), 1% N2 supplement (ThermoFisher, cat. no. 17502001), 2% B27 supplement (ThermoFisher, cat. no. 12587010) and a third small molecule compound combination. The parentheses represent the manufacturer and item number of the product.

Preferably, the third small molecule compound combination includes at least one of insulin, an agonist of RAR nuclear receptors, and an activator of the Hedgehog signaling pathway.

Preferably, the agonists and Hedgehog signaling pathway activators of the RAR nuclear receptor are consistent with the aforementioned.

In one embodiment, the OPC mature culture medium comprises a fourth small molecule compound combination.

In one embodiment, the OPC mature culture medium further includes a fourth basic culture medium, non-essential amino acids, glutamine, reducing agent, N2 supplement, and B27 supplement.

Preferably, the OPC mature culture medium also includes 95% DMEM/F-12 culture medium, 1% non-essential amino acid, 1% GlutaMAX-I, 2-Mercaptotothanol, 1% N2 supplement, and 2% B27 supplement.

Preferably, the OPC mature culture medium also includes 95% DMEM/F-12 culture medium (Life Technologies, cat. no. 11039021), 1% non-essential amino acid (Life Technologies, cat. no. 11140-050), 1% GlutaMAX-I (Life Technologies, cat. no. 35050061), 2-Mercaptotothanol (Gibco, 31350010), 1% N2 supplement (Thermo Fisher, cat. no. 17502001), and 2% B27 supplement (Thermo Fisher, cat. no. 12587010). The parentheses represent the manufacturer and item number of the product.

Preferably, the fourth small molecule compound combination includes at least one of insulin, PDGF-AA, IGF-1, HGF, NT3, T3, Biotin, and cAMP.

Preferably, the fourth small molecule compound combination includes insulin, PDGF-AA (PeproTech, cat. no. AF-100-13A), IGF-1 (PeproTech, cat. no. AF-100-11), HGF (PeproTech, cat. 100-39H), NT3 (PeproTech, cat. 450-03), T3 (Sigma Aldrich, cat. no. T2877), Biotin (Sigma Aldrich, cat. no. B4639), cAMP (Sigma Aldrich, cat. no. D0260), and 5-9 HT2C receptor antagonist. The parentheses represent the manufacturer and item number of the product.

Preferably, the fourth small molecule compound combination also contains a 5-HT2C receptor antagonist.

Preferably, the 5-HT2C receptor antagonists include Puerarin (puerarin), Harmine, SCH-23390 hydrochloride, Olanzapine, GTS-21 hydrochloride, Thioridazine hydrochloride, Risperidone, SB-269970 hydrochloride, Dihydroergotamine mesylate, Amitriptyline hydrochloride, Aripiprazole, Ketanserin, WAY-100635 Male, Buspirone hydrochloride, Cisapri de, Methiothepin mesylate Trazodone hydrochloride, Pindol, Quetiapine, Lumiteperone sysylate, Perphenazine, 8-OH-DPAT, Levomepromazine, SB-224289 hydrochloride, Volinaserin, Ziprasidone, Paliperidone, Sertindole, Alprenolol hydrochloride, Asenapine marine.

Preferably, the 5-HT2C receptor antagonist is Purerarin; preferably, its article number is medchem express, HY-N0145.

On the other hand, the present invention provides a method for inducing neural stem cells, which includes cell culture using the aforementioned neural induction complete culture medium and/or N2 culture medium.

The method comprises the aforementioned neural induction complete culture medium and the aforementioned N2 culture medium for cell culture.

Combination of small molecule compounds

On the other hand, the present invention also provides a small molecule compound combination comprising a 5-HT2C receptor antagonist.

Preferably, the small molecule compound combination is the fourth small molecule compound combination mentioned above.

### Test Kit

On the other hand, the present invention also provides a test kit for inducing oligodendrocytes, which includes one or more of the aforementioned insulin, TGF-β signaling pathway inhibitors, BMP signaling pathway inhibitors, RAR nuclear receptor agonists, Hedgehog signaling pathway activators, and 5-HT2C receptor antagonists.

Preferably, the test kit includes reagents configured with at least one of the following culture media:
1) Neural induction complete culture medium;
2) N2 culture medium;
3) B27 culture medium;
4) OPC mature culture medium.

### Application

On the other hand, the present invention provides any of the following culture media and their application in inducing oligodendrocytes:
1) Neural induction complete culture medium;
2) N2 culture medium;
3) B27 culture medium;
4) OPC mature culture medium.

On the other hand, the present invention provides the application of small molecule compound combinations containing 5-HT2C receptor antagonists in inducing oligodendrocytes.

On the other hand, the present invention provides the application of purerarin in inducing oligodendrocytes.

On the other hand, the present invention provides the application of the aforementioned test kit in inducing oligodendrocytes.

On the other hand, the present invention provides application of any one of insulin, TGF-β signaling pathway inhibitors, BMP signaling pathway inhibitors, RAR nuclear receptor agonists, Hedgehog signaling pathway activators, and 5-HT2C receptor antagonists in inducing oligodendrocytes.

On the other hand, the present invention provides application of insulin, TGF-β signaling pathway inhibitors, BMP signaling pathway inhibitors, RAR nuclear receptor agonists, and Hedgehog signaling pathway activators in inducing neural stem cells.

### Cells and Applications Thereof

On the other hand, the present invention provides a cell population, characterized in that the cell population is selected from one of the following:
1) a cell population prepared by the aforementioned method of inducing neural stem cells, which includes cells expressing at least one of NKX2.2+, Nestin+, and PAX6+.

Preferably, the cell population cells include cells expressing Olig2+.
2) the cell population prepared by the aforementioned method of inducing oligodendrocytes accounts for at least 20%, preferably, at least 21%, 25%, 30%, 35%, and 36% of all cells in the cell population.

Preferably, the oligodendrocyte is a cell that expresses at least one of Olig2, Nkx2.2, O4, and MBP.

Preferably, the oligodendrocyte is a cell co expressing Olig2 and Nkx2.2; alternatively, the oligodendrocyte is a cell co expressing O4 and MBP.

On the other hand, the present invention provides the application of cells prepared through the aforementioned method of inducing oligodendrocytes in the treatment of central nervous system diseases.

Preferably, the cells are oligodendrocytes.

Preferably, the central nervous system diseases include but are not limited to neurodegenerative diseases, demyelinating diseases, epilepsy, brain injury, shock, dementia, glaucoma, regeneration after neurological injury, and psychiatric diseases.

Preferably, the neurodegenerative diseases include Alzheimer's disease, cerebellar atrophy, primary lateral sclerosis, spinal muscular atrophy, Parkinson's disease, Huntington's disease, Creutzfeldt's disease, bovine spongiform encephalopathy, ataxia telangiectasia, amyotrophic lateral sclerosis, and psychiatric disorders.

The “demyelinating disease” mentioned in the present invention refers to the damage to the myelin sheath that occurs after the formation of the myelin sheath. Demyelinating diseases are a group of diseases characterized by the loss of nerve myelin sheath and relatively mild involvement of neuronal cell bodies and axons.

Preferably, the demyelinating diseases include two categories: hereditary and acquired.

Preferably, the demyelinating diseases include stroke, multiple sclerosis, optic neuromyelitis, acute disseminated encephalomyelitis, diffuse sclerosis, concentric sclerosis, white matter malnutrition, central pontine myelinolysis, acute inflammatory demyelinating multiple neuropathy, chronic inflammatory demyelinating multiple neuropathy, and white matter encephalopathy caused by ischemic hypoxic diseases Subacute combined degeneration caused by nutritional deficiency disease, subacute sclerosing panencephalitis or progressive multifocal leukoencephalopathy caused by virus infection, diabetes neuropathy, neuropathy of systemic lupus erythematosus, adrenoleukodystrophy, white matter ablation disease, Pelizaeus Merzbach disease.

Preferably, the mental illness includes schizophrenia, depression, paranoia, anxiety disorder, obsessive-compulsive disorder, and phobia,

On the other hand, the present invention provides a method for treating the aforementioned neurological disease, which includes inducing oligodendrocytes using the aforementioned method.

### BRIEF DESCRIPTION OF DRAWINGS

FIG. 1 shows the immunofluorescence detection image on the 16th day; A: Olig2+, Nkx2.2+, B: olig2+, Nestin+, C: olig2+, PAX6+.

FIG. 2 shows the immunofluorescence test on the 33rd day.

FIG. 3 shows the statistical results of the percentage of oligodendrocytes obtained in OPC mature culture medium with or without the addition of puerarin.

FIG. 4 shows the immunofluorescence detection of OPC mature culture medium with or without the addition of puerarin.

### DETAILED DESCRIPTION

The following is a further explanation of the present invention in conjunction with embodiments. The following is only a preferred embodiment of the present invention and does not impose any other form of limitation on the present invention. Any technical personnel familiar with the profession may use the disclosed technical content to modify it into equivalent embodiments with the same changes. Any simple modifications or equivalent changes made to the following embodiments based on the technical essence of the present invention without departing from the content of the present invention scheme shall fall within the scope of protection of the present invention.

### Reagents Used in the Present Invention

| | | |
|---|---|---|
| Commonly | Phosphate buffer without | Life Technologies, |
| used | Ca2+ and Mg2+, | cat. no. 14190-250 |
| | Dulbecco's phosphate- | |
| | buffered saline, D-PBS | |
| reagent | 5% BSA | sigma, V900933 |
| | 4% Paraformaldehyde | sigma, P6148 |
| | 0.3% Triton X-100 | Sigma-Aldrich, cat. no. |
| | | T9284-100ML |
| Primary | OPC marking | Olig2, Nkx2.2 |
| antibody | NSC marking | Nestin, Pax6 |
| | Anti-Olig2 antibody | Abcam, Cat. no. 109186 |
| | Anti-Nkx2.2 antibody | Abcam, Cat. no. 187375 |
| | Anti-Nestin antibody | Abcam, Cat. no. 22035 |
| | Anti-PAX6 antibody | Abcam, Cat. no. 5790 |
| Secondary | Alexa Fluor ® | Abcam, Cat. no. 150108 |
| antibody* | 594-conjugated | |
| | donkey anti-mouse | |
| | Alexa Fluor ® | Abcam, Cat. no. 150073 |
| | 488-conjugated | |
| | donkey anti-rabbit | |
| | Nuclear marking | Abcam, Cat. no. 104139 |
| | Fluoroshield Mounting | |
| | Medium With DAPI | |
| Com- | Non-essential amino acid | Life Technologies, |
| position | | cat. no. 11140-050 |
| of culture | GlutaMAX-I | Life Technologies, |
| medium | | cat. no. 35050061 |
| | Insulin | Sigma 12643 |
| | SB431542 | medchemexpress, |
| | | HY-10431 |
| | LDN193189 | medchemexpress, |
| | | HY-12071 |
| | Vitamin A acid | medchemexpress, |
| | | HY-14649 |
| | β-mercaptoethanol | Gibco, 31350010 |
| | SAG | medchemexpress, |
| | | HY-12848 |
| | N2 supplement | ThermoFisher, |
| | | cat. no. 17502001 |
| | B27 supplement | ThermoFisher, |
| | | cat. no. 12587010 |
| | Puerarin | medchemexpress, |
| | | HY-N0145 |

Secondary antibody: Alexa Fluor fluorescence marked antibody corresponding to the primary antibody

### General Method: Immunofluorescence Staining Identification

Preparation of working fluid:
5 1. Prepared 10 ml of blocked serum diluent (5% BSA+0.5% Triton X-100+DPBS solution,

taking 10 ml as an example; i.e. added 500 μL normal 5% BSA and 100 μL 30% Triton X-100 to 9.4 ml of DPBS).
2. Prepared the primary antibody working solution: added the appropriate titer of the primary antibody to the blocked serum dilution solution (see the specific titer value in the primary antibody user manual).
3. Prepared the secondary antibody working solution: blocked the serum diluent and added the appropriate secondary antibody titer (see the specific titer value in the secondary antibody user manual).
4. Prepared 90% glycerol: diluted with DPBS.

The specific steps of immunofluorescence staining are as follows:

Cleaned with DPBS three times, 3 minutes per time, and fixed with 4% PFA at room temperature for 40 minutes. Cleaned with DPBS three times, 3 minutes per time. 0.5% TritonX-100, perforation for 15 minutes. 5% BSA+0.15% TritonX-100, blocked at room temperature for 1 hour. Preparation of PBST: DPBS+1% BSA+0.15% TritonX-100. The primary antibody was added, stay overnight at 4 degrees. Recycled the primary antibody solution and cleaned it with PB ST three times for 10 minutes each time. The secondary antibody was added with a ratio of 1:500, stayed overnight at 4 degrees, away from light. Cleaned with PB ST three times for 10 minutes each time. 5 μg/ml DAPI for 2-3 minutes, away from light. Cleaned with DPBS and added 90% glycerol.

### Example 1: Inducing iPSC Cell Differentiation and Verifying the Induction Effect

Prepared the following culture medium for backup:

Neural induction complete medium: 98% DMEM/F-12 medium, 1% non-essential amino acid, 1% GlutaMAX-I, 0.1 mM 2-Mercaptotothanol, 10 μM SB431542, 0.25 μM LDN193189 and 100 μM Vitamin A acid, 25 μg/ml insulin.

N2 medium: 97% DMEM/F-12 medium, 1% non-essential amino acid, 1% GlutaMAX-I, 0.1 mM 2-Mercaptoethano, 1% N2 supplement, 1% μM SAG and 100 μM Vitamin A acid.

B27 medium: 95% DMEM/F-12 medium, 1% non-essential amino acid, 1% GlutaMAX-I, 0.1 mM 2-Mercaptotothanol, 1% N2 supplement, 2% B27 supplement, and 1 μM SAG and 100 μM vitamin A Acid, 25 μg/ml insulin.

OPC mature medium—without puerarin: 95% DMEM/F-12 medium, 1% non-essential amino acid, 1% GlutaMAX-I, 0.1 mM 2-Mercaptotothanol, 1% N2 supply, 2% B27 supply, 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 μg/ml insulin.

OPC mature medium—containing puerarin: 95% DMEM/F-12 medium, 1% non-essential amino acid, 1% GlutaMAX-I, 0.1 mM 2-mercaptoethanol, 1% N2 supplement, 2% B27 supplement, 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 μM Puerarin, 25 μg/ml insulin.

Followed the following steps to induce neural stem cells and verified their efficient differentiation into oligodendrocytes:
Started from day 0, iPSC was replaced with neural induction complete medium from E8 complete medium (STEMCELL, 05991).
2. Placed in a 37° C., 5% CO₂ incubator.
3. From the 1st to 7th day thereafter, changed the fluid daily.
4. Carefully observed the changes in cell morphology every day.
5. Started from day 8, the complete neural induction medium was replaced with N2 medium.
6. Placed in a 37° C., 5% CO₂ incubator.
7. From the 8th to 11th day thereafter, changed the fluid daily.
8. Carefully observed the changes in cell morphology every day.
9. Started from the 12th day, the N2 medium was replaced with B27 medium, and the cells were converted from adherent culture to suspension culture.
10. On the 12th day, remove the old culture medium and added B27 culture medium to

each well.
11. Used a sterilized blade to scrape the cells at least 20 times, then rotated the holes 90° and 45° respectively, and scraped at least 20 times each.
12. Used a cell scraper to scrape the entire hole along the scraping line and removed the cells.
13. Gently blew 3-5 times with a lml pipette, and then transferred 1 well of cells to the two wells of a low adsorption 6-well plate. Then added B27 culture medium to each well separately, so that the final volume of each well is 3m1; placed in a 5% CO₂ incubator at 37° C.
14. On the 12th to 19th days thereafter, changed the fluid every other day.
15. Carefully observed the changes in cell morphology every day.
16. Started from the 20th day, replaced B27 medium with OPC mature medium-without puerarin, and the cells will be converted from adherent culture to suspension culture.
17. On the 20th day, transferred the spherical aggregates into a 15 ml centrifuge tube using a 1 ml pipette, allow them to settle at the bottom of the centrifuge tube for 3 minutes, removed ⅔ of the old culture medium, and then added the same volume of OPC mature culture medium again. Then, transferred the spherical aggregates back into the original low adsorption 6-well plate.
18. On the 20th to 30th day thereafter, changed the fluid every other day.

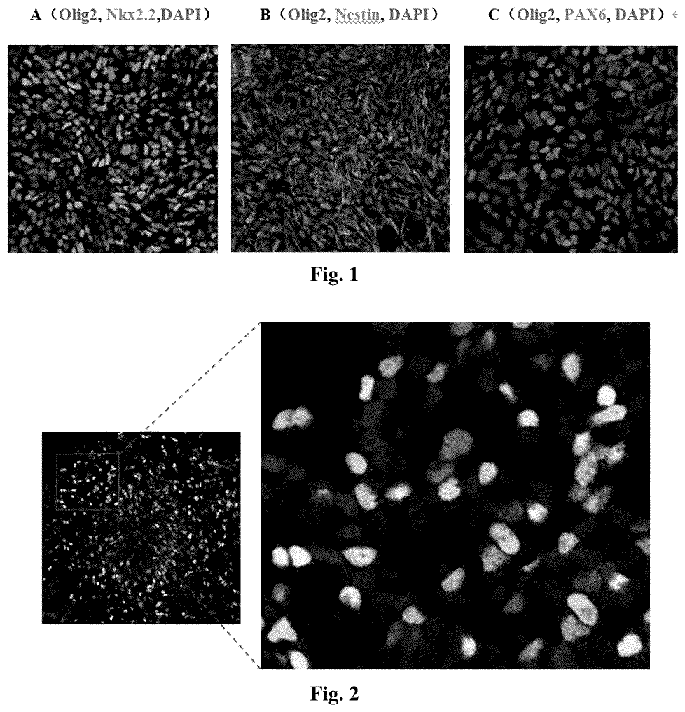

On the 16th day, immunofluorescence detection was performed using the general method, and the results are shown in FIG. 1:
A: There are both 01g2+(green, OPC marker) and NKX2.2+(red, neural stem cell marker)

cells present in the cells, but there are no cells expressing both Olg2+(green) and NKX2.2+(red), indicating that there are no mature OPC cells yet;
B: There are both Olg2+(green, OPC marker) and Nestin+(red, neural stem cell marker) positive cells present in the cells, but there are no cells expressing both Olg2+(green) and Nestin+(red), indicating that there are no mature OPC cells yet;
C: There are both Olg2+(green, OPC marker) and PAX6+(red, neural stem cell marker) cells present in the cells, but there are no cells that express both Olg2+(green) and PAX6+(red), indicating that there are no mature OPC cells yet.

Performed immunofluorescence testing according to the general method on the 33rd day; The results showed in FIG. 2 that Olig2 and Nkx2.2 were co expressed in cells after inducing day33, indicating a successful induction of OPC.

### Example 2: Effect of Puerarin on Induction of Oligodendrocyte in OPC Mature Culture Medium

Configured neural induction complete culture medium, N2 culture medium, B27 culture medium, and OPC mature culture medium according to the method of Example 1; and prepared OPC mature culture medium without puerarin; using OPC mature culture medium containing or without puerarin as a control, explore the effect of puerarin on inducing oligodendrocyte formation.

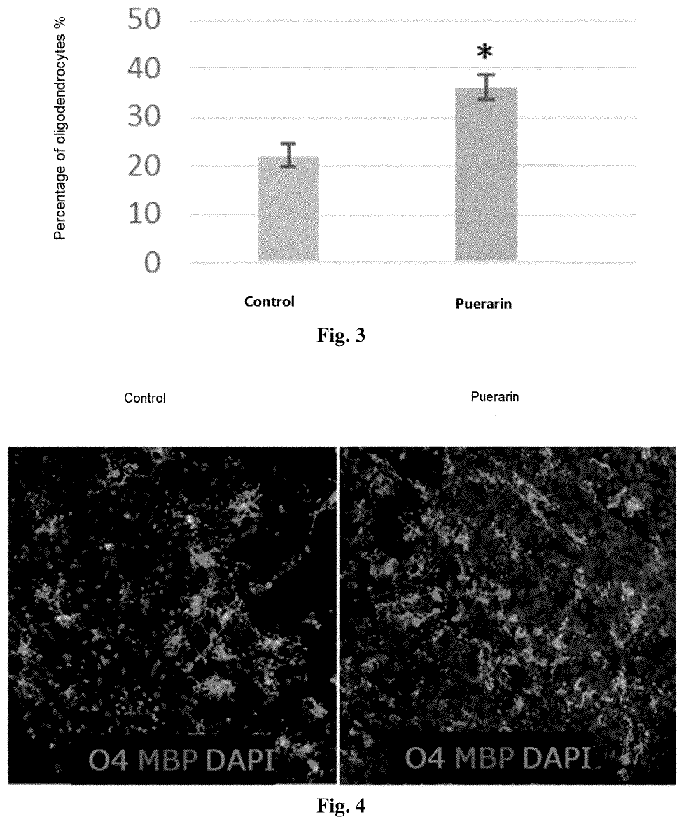

On the 37th day of cultivation, the percentage of oligodendrocytes in all cells was quantitatively counted, as shown in FIG. 3. Immunofluorescence analysis was performed, and the results are shown in FIG. 4.

Using OPC mature culture medium without puerarin as the control group, the control group was able to produce about 21% oligodendrocytes, and adding puerarin could produce about 36% oligodendrocytes. Prove that adding puerarin to OPC mature culture medium is beneficial for the production of oligodendrocytes.

1. A culture medium combination, wherein the culture medium combination consists of a neural induction complete culture medium, N2 culture medium, B27 culture medium and OPC mature culture medium;
the neural induction complete culture medium consists of a first basic culture medium, non-essential amino acids, glutamine, reducing agent and a first small molecule compound combination, the first small molecule compound combination consists of 10 μM SB431542, 0.25 μM LDN193189, 100 μM vitamin A acid and 25 μg/ml insulin;
the N2 culture medium consists of a second basic culture medium, non-essential amino acids, glutamine, reducing agent, N2 supplement, and a second small molecule compound combination; the second small molecule compound combination consists of 1 μM SAG and 100 μM vitamin A;
the B27 culture medium consists of a third basic culture medium, non-essential amino acids, glutamine, reducing agent, N2 supplement, B27 supplement and a third small molecule compound combination; the third small molecule compound combination consists of 1 μM SAG, 100 μM vitamin A acid and 25 μg/ml insulin;
the OPC mature culture medium consists of a fourth basic culture medium, non-essential amino acids, glutamine, reducing agent, N2 supplement, B27 supplement and a fourth small molecule compound combination; the fourth small molecule compound combination consists of 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 μM Purerarin, 25 μg/ml insulin.

2. The culture medium combination according to claim 1, the first basic culture medium, the second basic culture medium, the third basic culture medium and the fourth basic culture medium are each independently DMEM/F-12 culture medium.

3. The culture medium combination according to claim 1, the non-essential amino acids include alanine, arginine, aspartic acid, cystine, proline and tyrosine.

4. The culture medium combination according to claim 1, the glutamine used is GlutaMAX-I.

5. The culture medium combination according to claim 1, the reducing agent is β-mercaptoethanol.

6. The culture medium combination according to claim 1, the neural induction complete culture medium consists of 98% DMEM/F-12 culture medium, 1% non-essential amino acid, 1% GlutaMAX-I, 0.1 mM β-mercaptoethanol, 10 μM SB431542, 0.25 μM LDN193189 and 100 μM vitamin A acid, 25 μg/ml insulin.

7. The culture medium combination according to claim 1, the N2 medium consists of 97% DMEM/F-12 medium, 1% non-essential amino acid, 1% GlutaMAX-I, 0.1 mM β-mercaptoethanol, 1% N2 supplement, 1 μM SAG and 100 μM vitamin A acid.

8. The culture medium combination according to claim 1, the B27 culture medium consists of 95% DMEM/F-12 culture medium, 1% non-essential amino acid, 1% GlutaMAX-I, 0.1 mM β-mercaptoethanol, 1% N2 supply, 2% B27 supply, and 1 μM SAG and 100 μM vitamin A acid, 25 μg/ml insulin.

9. The culture medium combination according to claim 1, the OPC mature medium consists of 95% DMEM/F-12 medium, 1% non-essential amino acid, 1% GlutaMAX-I, 0.1 mM β-mercaptoethanol, 1% N2 supply, 2% B27 supply, 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 μM Purerarin, 25 μg/ml insulin.

10. A method for inducing human induced pluripotent stem cells to form oligodendrocytes, comprising culturing on days 0-7 using the neural induction complete medium according to claim 1, culturing on days 8-11 using the N2 medium according to claim 1, culturing on days 12-19 using the B27 medium according to claim 1, and culturing on days 20-30 utilizing the OPC mature medium according to claim 1.

11. An application of the culture medium combination according to claim 1 in inducing human induced pluripotent stem cells to form oligodendrocytes.
